(19) United States
(12) Patent Application Publication
(10) Pub. No.: US 2005/0026306 A1
(43) Pub. Date: Feb. 3, 2005

(54) Method and system for generating virtual-microarrays

(76) Inventors: Robert Kincaid, Half Moon Bay, CA (US); Paul K. Wolber, Los Altos, CA (US)

Correspondence Address: AGILENT TECHNOLOGIES, INC.;Legal Department, DL 429, Intellectual Property Administration, P.O. Box 7599, Loveland, CO, 80537-0599, US

(21) Appl. No.: 10/633,470

(22) Filed: Jul. 31, 2003

(51) Int. Cl. C12M 1/34; G01N 33/543

(52) U.S. Cl. 436/518; 435/287.2

(57) ABSTRACT

A method and system for generating virtual-microarray feature data from a virtualizing catalog array comprising a catalog microarray associated with data that can be together processed to produce any of numerous sets of virtual-microarray feature data. The catalog array portion of the virtualizing microarray may include many thousands, tens of thousands, or hundreds of thousands of different features from which a very large number of feature subsets may be generated. The data associated with the virtualizing microarray allows for rapid and transparent partitioning of the catalog-microarray features. Logic included within a microarray scanner, a computer system used to process data scanned from microarrays, a microarray-data visualization system, or other microarray-related processing entities can be used to generate feature data for any number of user-specified virtual arrays based on partitioning of the features included in the catalog-microarray component of the virtualizing microarray.

### TECHNICAL FIELD

The present invention is related to microarrays and, in particular, to a method and system for generating feature data associated with user-specified virtual microarrays from a combination of a traditional catalog microarray and associated data and logic.

### BACKGROUND OF THE INVENTION

The present invention is related to microarrays. A general background of microarray technology is first provided, in this section, to facilitate discussion of the scanning techniques described in following sections. Microarrays are also referred to as “molecular arrays” and simply as “arrays” in the literature. Microarrays are not arbitrary regular patterns of molecules, such as occur on the faces of crystalline materials, but, as the following discussion shows, are manufactured articles specifically designed for analysis of solutions of compounds of chemical, biochemical, biomedical, and other interests.

Array technologies have gained prominence in biological research and are likely to become important and widely used diagnostic tools in the healthcare industry. Currently, microarray techniques are most often used to determine the concentrations of particular nucleic-acid polymers in complex sample solutions. Microarray-based analytical techniques are not, however, restricted to analysis of nucleic acid solutions, but may be employed to analyze complex solutions of any type of molecule that can be optically or radiometrically scanned or read and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrates the hydrogen bonding between the purine and pyrimidine bases of two anti-parallel DNA strands. AT and GC base pairs, illustrated in FIGS. 2A-B, are known as Watson-Crick (“WC”) base pairs. Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for data acquisition by scanning or reading. Biotin and avidin represent an example of a pair of chemical moieties that can be utilized for such steps.

Finally, as shown in FIG. 6, the bound labeled DNA molecules are detected via optical or radiometric scanning or reading. Optical scanning and reading both involve exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning or reading can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning and reading produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

One, two, or more than two data subsets within a data set can be obtained from a single microarray by scanning or reading the microarray for one, two or more than two types of signals. Two or more data subsets can also be obtained by combining data from two different arrays. When optical scanning or reading is used to detect fluorescent or chemiluminescent emission from chromophore labels, a first set of signals, or data subset, may be generated by scanning or reading the microarray at a first optical wavelength, a second set of signals, or data subset, may be generated by scanning or reading the microarray at a second optical wavelength, and additional sets of signals may be generated by scanning or reading the molecular at additional optical wavelengths. Different signals may be obtained from a microarray by radiometric scanning or reading to detect radioactive emissions one, two, or more than two different energy levels. Target molecules may be labeled with either a first chromophore that emits light at a first wavelength, or a second chromophore that emits light at a second wavelength. Following hybridization, the microarray can be scanned or read at the first wavelength to detect target molecules, labeled with the first chromophore, hybridized to features of the microarray, and can then be scanned or read at the second wavelength to detect target molecules, labeled with the second chromophore, hybridized to the features of the microarray. In one common microarray system, the first chromophore emits light at a red visible-light wavelength, and the second chromophore emits light at a green, visible-light wavelength. The data set obtained from scanning or reading the microarray at the red wavelength is referred to as the “red signal,” and the data set obtained from scanning or reading the microarray at the green wavelength is referred to as the “green signal.” While it is common to use one or two different chromophores, it is possible to use one, three, four, or more than four different chromophores and to scan or read a microarray at one, three, four, or more than four wavelengths to produce one, three, four, or more than four data sets.

FIG. 8 illustrates two different, commonly used approaches for employing microarrays to collect data for particular experiments. As shown in FIG. 8, in a particular experiment, a list 802 of particular probe molecules, or equivalently, a list of particular target molecules for which corresponding probe molecules need to be included in a microarray, is used to specify a microarray needed for the experiment. In FIG. 8, the probe-molecule identities are illustrated as two-character alpha-numeric strings, such as the string “a1” in the first entry of the list 802. In one commonly used approach, a traditional catalog microarray 804 that includes the probe molecules specified in the list 802 is selected for use in the experiment. A catalog microarray may include tens of thousands or more of different probe molecules, and is designed and manufactured in order to be generally useful in a wide variety of different experiments. However, in a particular experiment, a user must carefully identify those features in the catalog corresponding to the specified probe molecules in the list. Moreover, following exposure of the catalog array to one or more sample solutions, the catalog array needs to be processed in order to provide properly normalized feature data for the specified probe molecules. This processing may require sophisticated, specialized feature extraction and bioinformatics analysis in order to provide feature extraction and normalization suitable for the subset of features of the catalog array relevant to the experiment. As often practiced, the tasks associated with extracting data particular to a given experiment from a general, catalog microarray are carried out on an ad hoc basis, and are therefore relatively time consuming.

A second, commonly used approach for experimental design is to design and build a custom microarray based on the probe molecules directly or indirectly specified in the list 802. As shown in FIG. 8, the list of probe molecules 802 may be rather directly translated into a block of features 806 within a customized microarray 808. However, custom microarrays also have drawbacks. First, it is non-trivial and relatively expensive to design and manufacture specialized microarrays for particular experiments. Second, customized microarrays may be somewhat inefficient. For example, consider the customized microarray 808 in FIG. 8. In this example, only a small fraction of the potential number of features is used. Redundant features, control features, and other additional features may be included in the custom microarray, in order to better use the potential capacity of a custom microarray, but including the additional features involves additional design and manufacturing efforts. More importantly, the design and manufacture of a custom microarray may involve a significant expenditure in time, delaying an experiment and the acquisition of experimental results. Thus, there is a need for a more time-and-cost-effective method for employing microarrays in experiments and for experimental design related to microarrays.

### SUMMARY OF THE INVENTION

One embodiment of the present invention is a method and system for generating virtual-microarray feature data from a virtualizing catalog array comprising a catalog microarray associated with data that can be together processed to produce any of numerous sets of virtual-microarray feature data. The catalog array portion of the virtualizing microarray may include many thousands, tens of thousands, or hundreds of thousands of different features from which a very large number of feature subsets may be generated. The data associated with the virtualizing microarray allows for rapid and transparent partitioning of the catalog-microarray features. Logic included within a microarray scanner, a computer system used to process data scanned from microarrays, a microarray-data visualization system, or other microarray-related processing entities can be used to generate feature data for any number of user-specified virtual arrays based on partitioning of the features included in the catalog-microarray component of the virtualizing microarray. Thus, the virtualizing microarray can be used to relatively transparently generate any of numerous user-specified virtual microarrays in a time-and-cost-efficient manner.

### BRIEF DESCRIPTION OF THE DRAWINGS

FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108.

FIGS. 2A-B illustrate the hydrogen bonding between the purine and pyrimidine bases of two anti-parallel DNA strands.

FIG. 3 illustrates a short section of a DNA double helix 300 comprising a first strand 302 and a second, anti-parallel strand 304.

FIGS. 4-7 illustrate the principle of the array-based hybridization assay.

FIG. 8 illustrates two different, commonly used approaches for employing microarrays to collect data for particular experiments.

FIG. 9 provides a graphical overview of one embodiment of the present invention.

FIG. 10 abstractly illustrates a description of a catalog-array feature that may be included within the data associated with the catalog-array component of a virtualizing microarray.

FIG. 11 illustrates a number of components of a microarray scanning, data extraction, data processing, and data visualization system in which a virtualizing microarray may be employed.

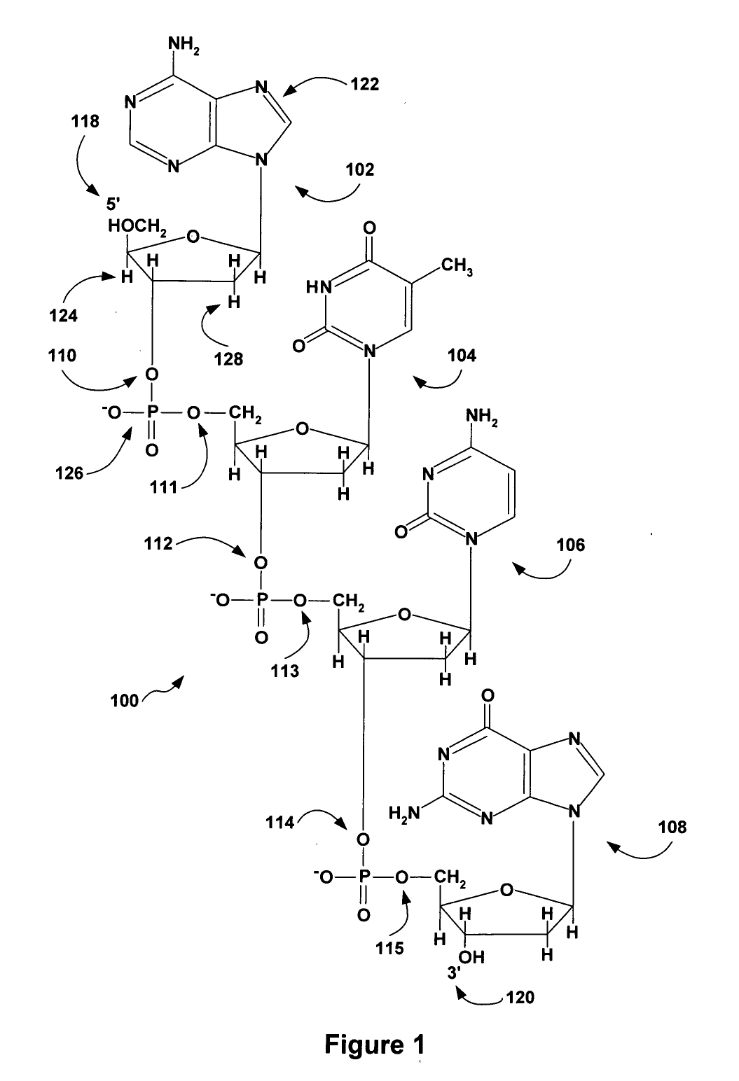

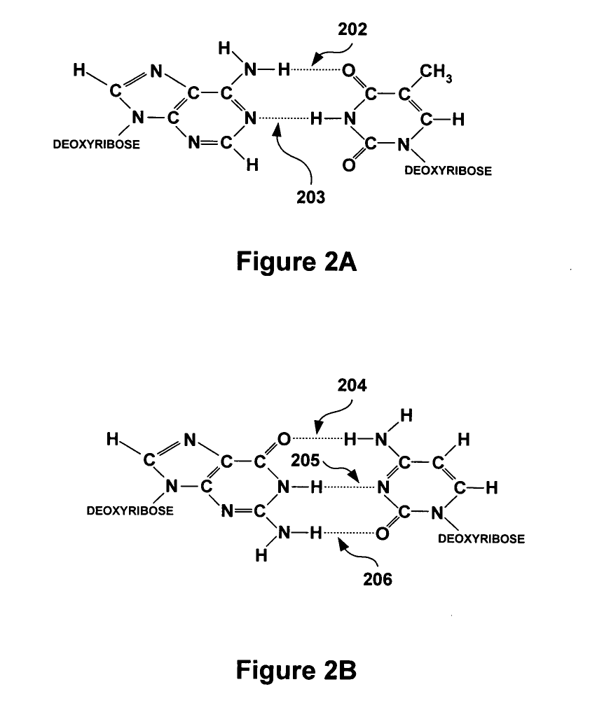

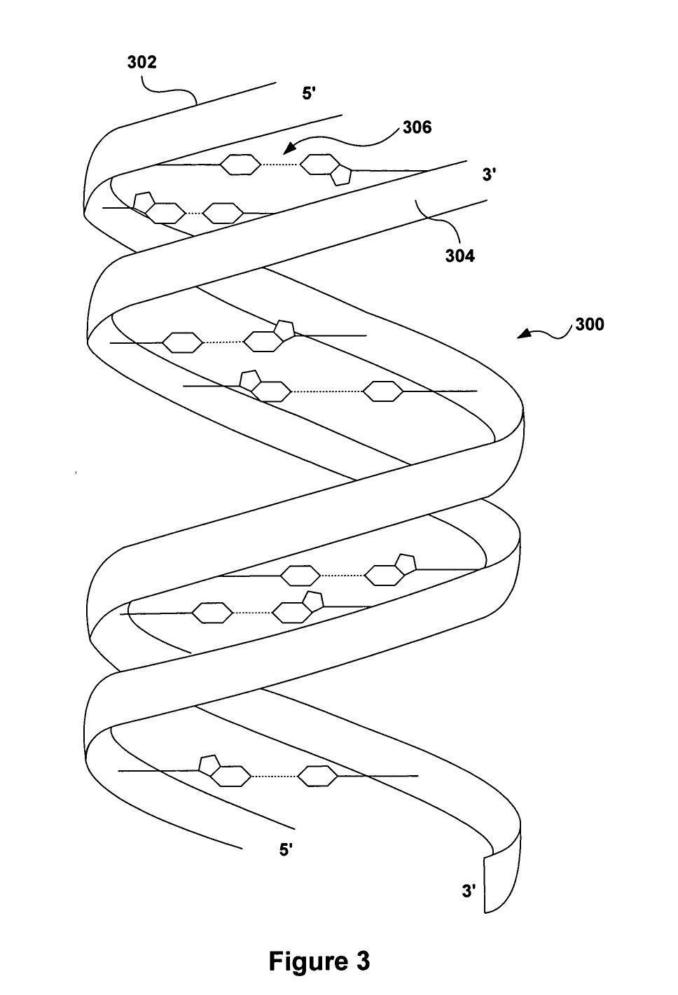

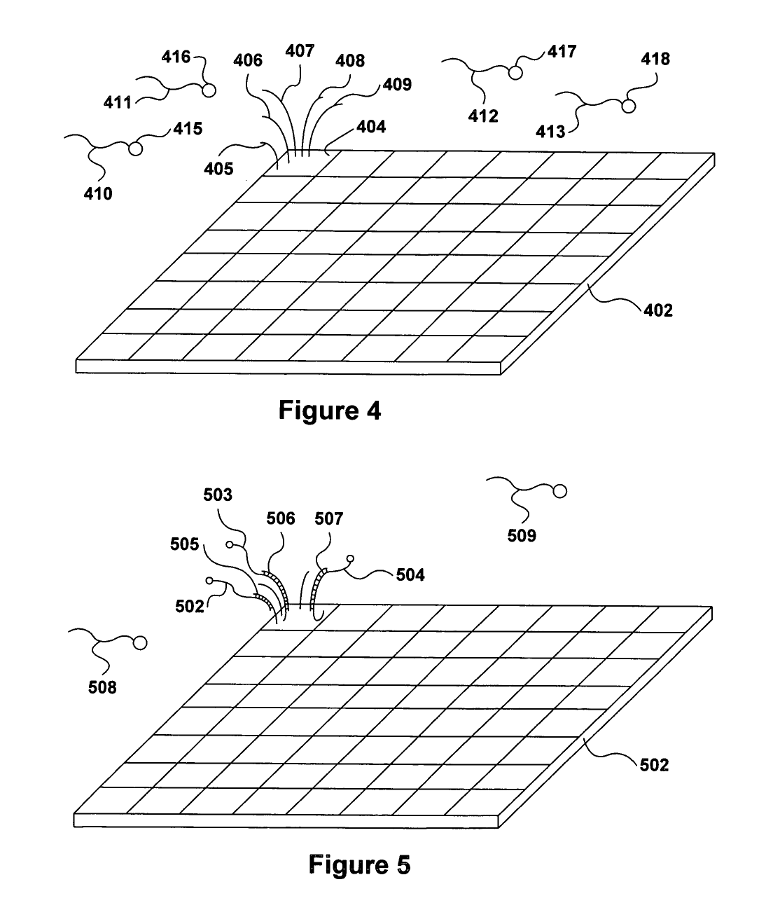

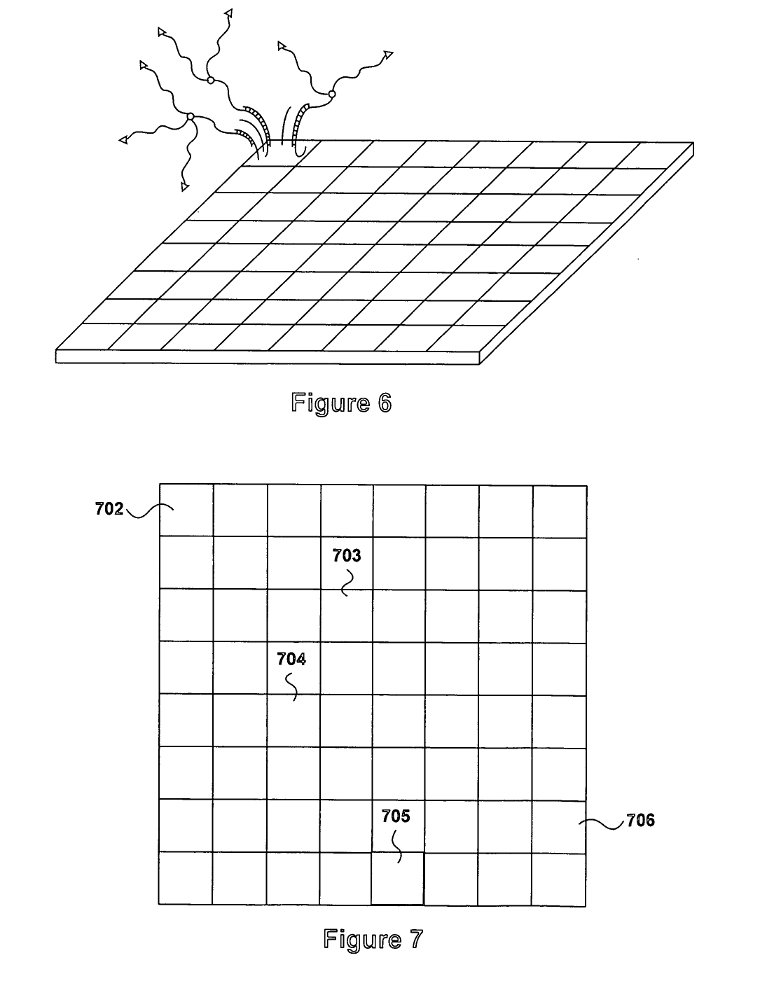

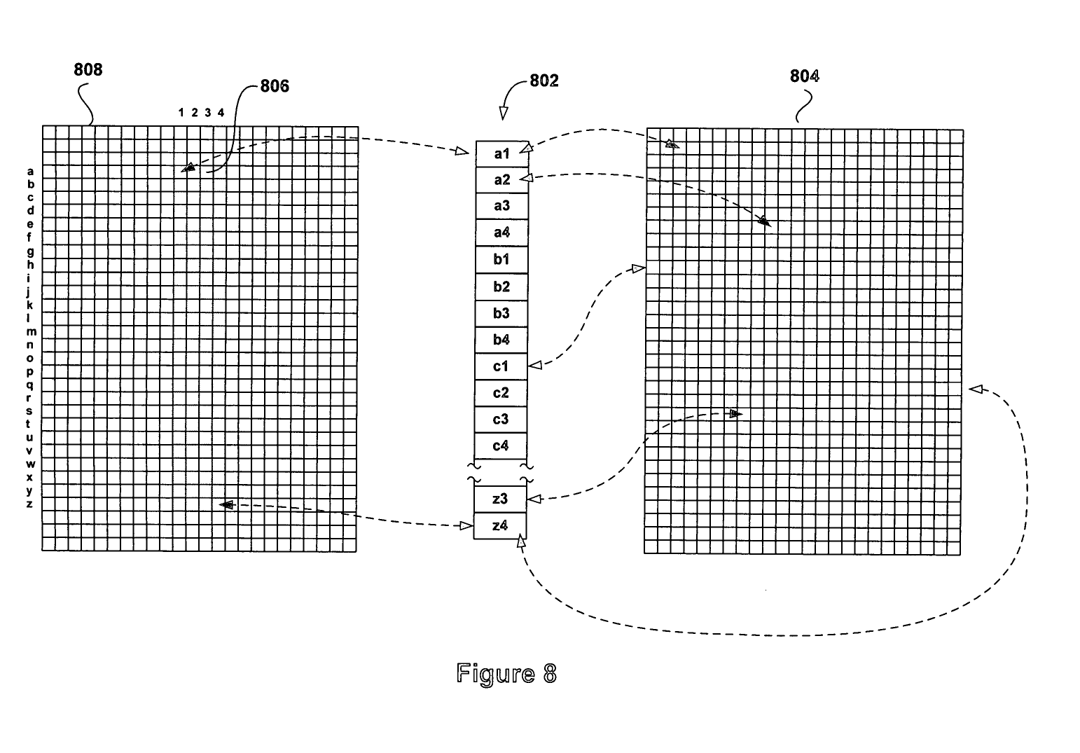

### DETAILED DESCRIPTION OF THE INVENTION

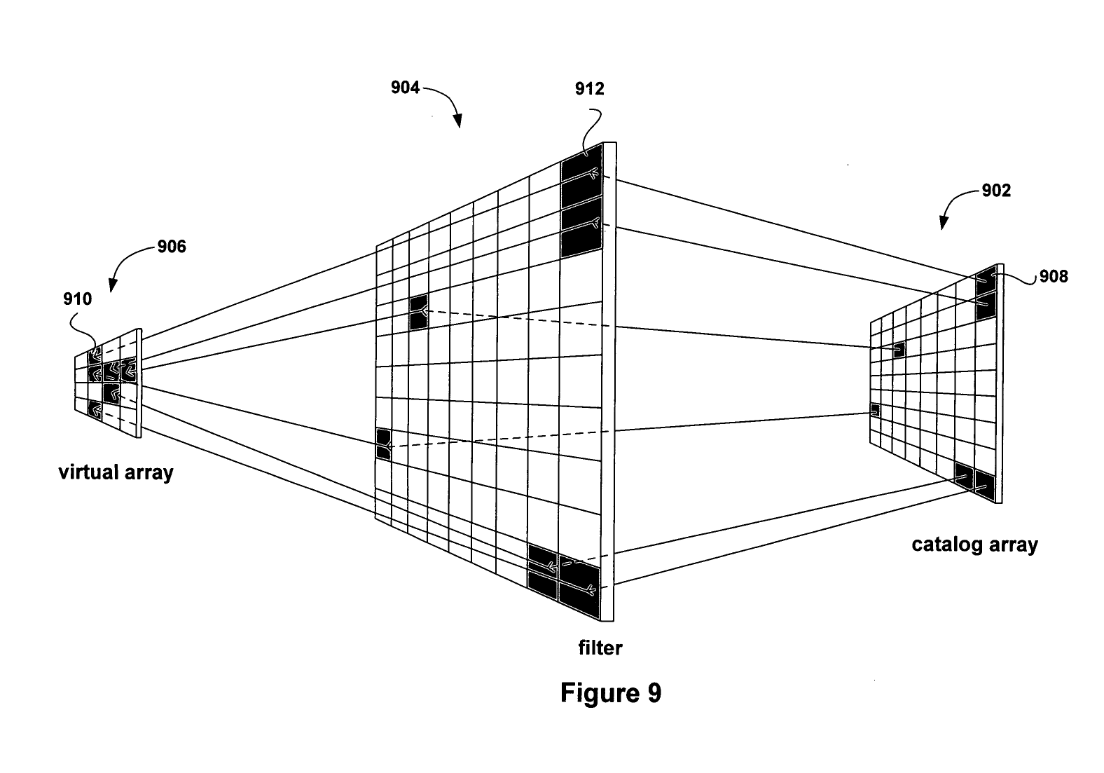

One embodiment of the present invention provides a virtualizing-microarray method and system that enables transparent and time-and-space-efficient generation of numerous user-specified virtual-microarray data sets. The present invention is described, below, in three subsections. A first subsection provides additional information about microarrays. A second subsection provides an overview of one embodiment of the present invention with reference to FIGS. 9-11. A third subsection provides a C++-like pseudocode implementation of a virtualizing microarray.

### Additional Information About Microarrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Interfeature areas are typically, but not necessarily, present. Interfeature areas generally do not carry probe molecules. Such interfeature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, known photolithographic array fabrication methods may be used. Interfeature areas need not be present particularly when the arrays are made by photolithographic methods as described in those patents.

A microarray is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications: Ser. No. 10/087,447 “Reading Dry Chemical Arrays Through The Substrate” by Corson et al., and Ser. No. 09/846,125 “Reading Multi-Featured Arrays” by Dorsel et al. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, U.S. Pat. No. 6,221,583 and elsewhere.

A result obtained from reading an array may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based microarray, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a microarray by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Microarray experiments can indicate precise gene-expression responses of organisms to drugs, other chemical and biological substances, environmental factors, and other effects. Microarray experiments can also be used to diagnose disease, for gene sequencing, and for analytical chemistry. Processing of microarray data can produce detailed chemical and biological analyses, disease diagnoses, and other information that can be stored in a computer-readable medium, transferred to an intercommunicating entity via electronic signals, printed in a human-readable format, or otherwise made available for further use.

### Overview of One Embodiment of the Present Invention

FIG. 9 provides a graphical overview of one embodiment of the present invention. A virtualizing microarray that represents an embodiment of the present invention comprises a traditional catalog microarray 902 and data 904 that, combined with logic in a microarray scanner, microarray-data-processing system, microarray-data-visualization system, or other microarray-related processing entity, acts as a filter or map to map data scanned from features of the catalog array 902 onto a virtual microarray 906. The data 904 associated with the catalog array, in combination with processing logic, such as a software program, allows a user to transparently view data scanned from the catalog array 902 as virtual-array data. Virtual-array data may be input into feature-extraction and microarray-data-processing programs in order to generate normalized virtual-array feature data, without the need for complex, ad hoc catalog-array-data processing. As shown in FIG. 9, the data 904 and logic can map a particular feature, such as feature 908, to any specified feature of a virtual array, feature 910 in the illustrated example, via a description of the catalog-array feature 912 included in the data 904.

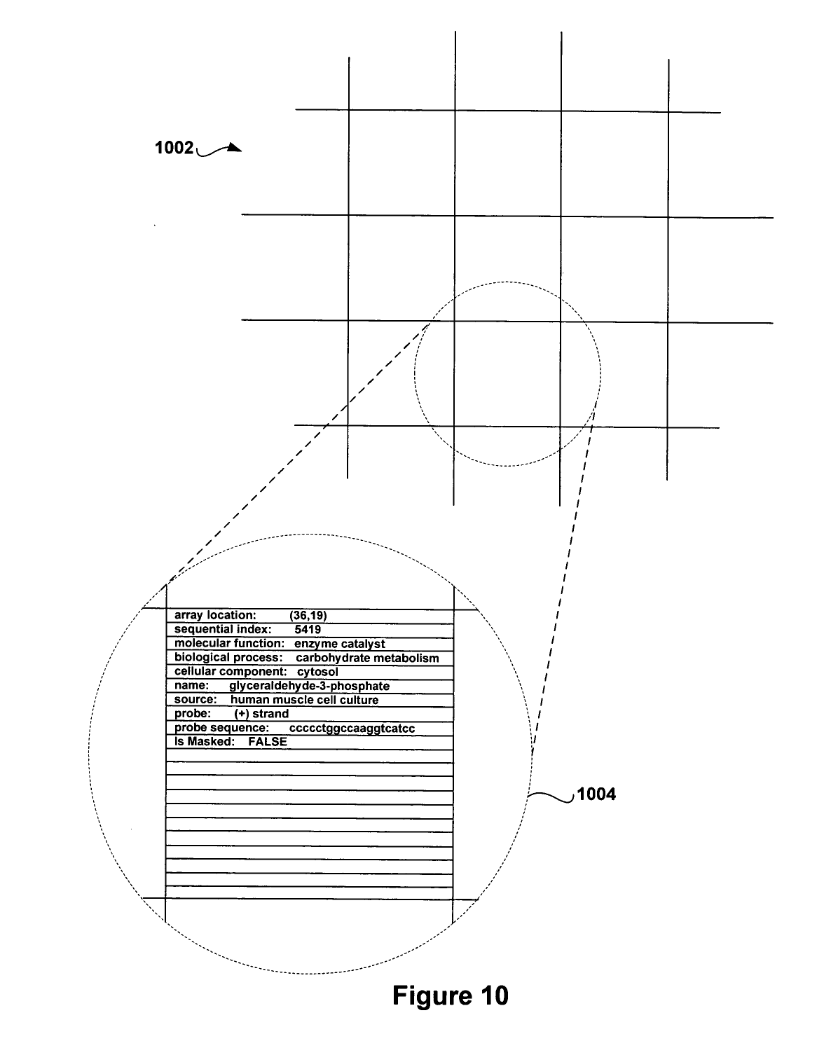

FIG. 10 abstractly illustrates a description of a catalog-array feature that may be included within the data (904 in FIG. 9) associated with an oligonucleotide-based catalog-array component of a virtualizing microarray. As shown in FIG. 10, the data may be considered to comprise a two-dimensional grid of records 1002, each record 1004 comprising a list of fields containing data values. In the example shown in FIG. 10, a record 1004 is a flat list of data-containing fields, but in alternative embodiments, more complex, hierarchically organized records, or objects, may be employed. In the example record 1004 shown in FIG. 10, fields include: (1) “array location,” the coordinates in a two-dimensional, grid-like coordinate system of the feature described by the record; (2) “sequential index,” a sequential index of the feature when the two-dimensional grid of features has collapsed into a one-dimensional list, row-by-row; (3) “molecular function,” a short description of the molecular function of the gene product of the mRNA targeted by the probe; (4) “biological process,” the overall biological process that the translational product of the mRNA targeted by the probe is involved in; (5) “cellular component,” the cellular component in which the translational product of the mRNA targeted by the probe normally resides; (6) “name,” the name for the translational product of the mRNA targeted by the probe; (7) “source,” the biological source of the target to which the probe is directed; (8) an indication of whether a probe targets the plus or minus strand of a gene; (9) “probe sequence,” the nucleotide sequence of the probe molecule; and (10) and a Boolean field indicating whether or not the probe is masked for inclusion in, or exclusion from, a virtual array. In certain implementations, a large number of Boolean fields may be used to filter features for inclusion into different virtual microarrays. In other embodiments, filtering may occur based on non-Boolean-field values, such as the values in the first nine fields of the exemplary record shown in FIG. 10, and in still other embodiments, a combination of Boolean and non-Boolean-field filters may be employed. The example data record assumes that target molecules are mRNA molecules that direct translation of protein products. However, a richer set of data fields may be employed to include rRNAs, tRNAs, dsRNAs, ribozymes, and other RNA products of gene transcription.

The data component of a virtualizing microarray needs to be at least tightly conceptually associated with a particular type of catalog microarray. More desirable is a physical association between the data component and the catalog-microrarray components of a virtualizing microarray. For example, a physical microarray may include a small microchip-memory that can be electronically queried to provide for a self-describing catalog-array component. Alternatively, the catalog microarray may include a smaller, electronic data-storage device from which an identification number and/or alpha-numeric descriptive string can be obtained in order to match the catalog array with a corresponding data component within a data-processing system.

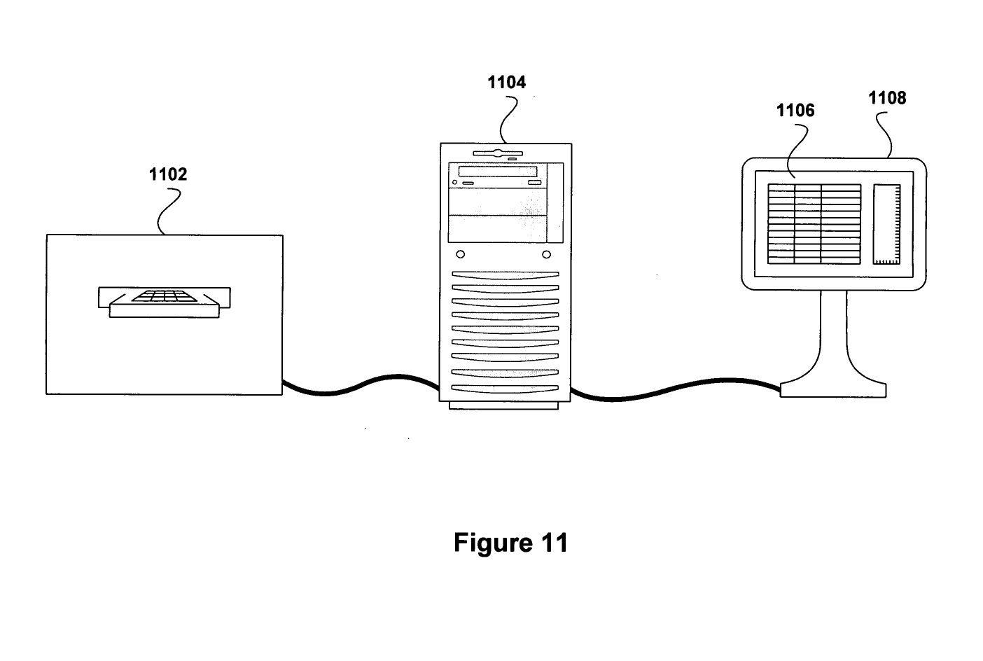

FIG. 11 illustrates a number of components of a microarray scanning, data extraction, data processing, and data visualization system in which a virtualizing microarray may be employed. The virtualizing microarray that represents one embodiment of the present invention may be employed at any one or more of many different steps in microarray-data acquisition and display. For example, as shown in FIG. 11, microarrays, following exposure to sample solutions, are scanned in a microarray scanner 1102. The microarray scanner may include logic, in the form of firmware, software, or a combination of firmware and software, for transforming signal intensities generated by the optical components within a scanner into raw numeric data, each pixel within the scanned image of a feature of the surface of a microarray associated with a numeric intensity. The pixel-based intensity data is then processed, either within the scanner, or within a data-processing component, such as a personal computer or workstation 1104, interconnected with the microarray scanner. The pixel-intensity data is processed to produce a normalized signal intensity and background intensity for each feature of the microarray. These intensities can then be further processed in order to produce textual, graphical, or a combination of textual and graphical displays 1106 of the data on a display device 1108. The data component of a virtualizing microarray may be accessed by logic within a microarray scanner 1102, a data-processing system 1104, or a data-display generation component (in the example in FIG. 11, also 1104). Thus, a virtualizing microarray may be transformed into a virtual microarray at any or at multiple points within the microarray-processing component stream. For example, when employed in the scanner, only those features relevant to a user-specified virtual microarray may be scanned, and processed downstream from the scanner. Alternatively, first introduction of a virtualizing microarray at the feature extraction or normalization steps may be used to filter scanned data to produce only that data relevant to a particular virtual microarray.

A virtualizing microarray allows a single catalog microarray to be conveniently used as any of a huge number of user-specified virtual microarrays. Each virtual microarray represents a subset of data related to features of the catalog microarray that can be treated computationally as data related to a different, smaller microarray. In other words, by using a virtualizing microarray, a researcher can specify a custom virtual array needed for a particular experiment, and obtain the data by using a standard, catalog array. The virtual microarray is nearly transparently obtained by computational methods, one example of which is provided below. The researcher neither needs to design a custom, physical microarray, nor needs to develop ad hoc methods for extracting the needed data from a catalog microarray. Moreover, the virtual microarray can be made available at each step in the microarray scanning, data processing, and data visualization stages.

### One Embodiment of the Present Invention

In this subsection, a brief pseudocode implementation of an embodiment of the data component and associated logic for a virtualizing microarray is provided. This C++-like pseudocode implementation is provided merely as an illustration of one possible approach for implementing the data component and associated logic for a virtualizing microarray. The pseudocode implementation omits detailed error detection and correction and other features commonly included in commercial implementations, in the interest of clarity and brevity. Note that there are an almost limitless number of different ways for implementing even the simplest logic component, including different modular structures, data structures, control structures, programming languages, and other such parameters and characteristics.

First, several constants are defined:
1 const int MAX_NAME=100;
2 const int MAX_VALS=50;
3 const int MAX_ROWS=100;
4 const int MAX_COLUMNS=100;
   The constant “MAX_NAME” declared above on line 1, is the maximum number of characters allowed in a text string that represents the value of a field within a record in the data component that describes a feature. The constant “MAX_VALS” declared above on line 2, indicates the maximum number of values that any particular text field may have. The constants “MAX_ROWS” and “MAX_COLUMNS,” declared above on lines 3-4, indicate the maximum numbers of rows and columns within a catalog array. Note that these constant values have been set to small values adequate only for an exemplary pseudocode implementation.

Next, the class “stringValCategory” is declared below:

```
  1 class stringValCategory
  2 {
  3  private:
  4  char vals[MAX_VALS][MAX_NAME];
  5  bool masks[MAX_VALS];
  6  int num;
  7  bool anyMask;
  8
  9  public:
10  int addVal(const char* val);
11  int getIndex(const char* val);
12  char* getString(int index);
13  int setMaskOn(const char* val);
14  int setMaskOff(const char* val);
15  bool mask(int dex);
16  void clearMasks( );
17  stringValCategory( );
18 };
```

The class “stringValCategory” contains the different values that may be assigned to a particular field within a record that describes a feature. Each value is also associated with a Boolean mask flag, which may be set ON or OFF to indicate whether or not the particular value should serve as a positive mask or filter for selecting features for a virtual microarray. Thus, for example, if the value “carbohydrate metabolism” for the field “biological process” is associated with a mask flag set to the value ON or TRUE, then, when a virtual microarray is created, features of the catalog microarray for which the field “biological process” has the value “carbohydrate metabolism” will be included in the virtual microarray. A field for which no values have the Boolean mask flag set does not participate in the masking or filtering process. The class “stringValCategory” contains the following private data members, declared above on lines 4-7: (1) “vals,” declared above on line 4, an array of text strings that represent the different possible values for a particular field; (2) “masks,” an array of Boolean mask flags, each flag associated with a corresponding field value in the above-described array vals; (3) “num,” the number of values defined for the field; and (4) “anyMask,” a Boolean flag indicating whether or not the field participates in the masking or filtering process by which virtual microarrays are generated. The class “stringValCategory” includes the following public function members, declared above on lines 10-17: (1) “addVal,” a function member that adds a text-string value to the list of values for the field represented by an instance of the class “stringValCategory,” (2) “getIndex,” a function member that returns a numeric index of a value provided by the argument “val,” the returned index equivalent to the position of the value within the private data member “vals;” (3) “getString,” a function member that returns the text-string value associated with a particular numerical index into the private data member “vals;” (4) “setMaskOn” and “setMaskOff,” functions members that turn on and off, respectively, the Boolean mask flag associated with a field value supplied as argument “val;” (5) “mask,” a member function that returns a Boolean value indicating whether or not the value represented by the supplied index “dex” is a positive mask; (6) “clearMasks,” a function member that clears all Boolean mask flags for an instance of the class “stringValCategory”; and (7) a constructor for the class “stringValCategory.”

Next, two dummy class declarations are provided, one for a class representing a header that may describe a particular category microarray, and one for the data that is stored for each feature in the data component of a virtualizing microarray:

class arrayHeader
{
};
class data
{
};

An array header may include an identification number, an identifying text string, or other such information. It may also include a date of manufacture, a version number, and other types of product information. An instance of the class “data” may include raw pixel-based intensity data, averaged background and region-of-interest intensity data, a normalized numeric intensity value, and other such scanned-data-related information. Because, in both cases, the precise nature of the member data and member functions are unnecessary for describing an exemplary implementation of one embodiment of the present invention, these details are omitted. Next, following two initial class-name declarations, a declaration for the class “arrayElement” is provided:

```
class arrayElement;
class catalogArray;
  1 class arrayElement
  2 {
  3  friend class catalogArray;
  4
  5  private:
  6  data dt;
  7  int molecularFunction;
  8  int bioProcess;
  9  int cellularComp;
10  int name;
11  int source;
12  int strand;
13  int sequence;
14  arrayElement* next;
15
16  arrayElement* getNext( );
17  void  setNext(arrayElement* nxt);
18
19  public:
20  int getMolecularFunction( );
21  void setMolecularFunction(int dex);
22  int getBioProcess( );
23  void setBioProcess(int dex);
24  int getCellularComp( );
25  void setCellularComp(int dex);
26  int getName( );
27  void setName(int dex);
28  int getSource( );
29  void setSource(int dex);
30  int getSequence( );
31  void setSequence(int dex);
32  data getData( );
33  void setData (const data d);
34  arrayElement( );
35 };
```

The class “catalogArray” represents a data record corresponding to a feature of a catalog array. The class “arrayElement” includes, in the illustrative implementation, the following private data members, declared above on lines 6-14: (1) “dt,” the scanned-feature data described by an instance of the class “data;” (2) “molecular function,” the index of a text-string value describing the molecular function of the product of the target mRNA for the probe of the feature described by an instance of the class “arrayElement;” (3) indexes for the text-string values for fields that describe the biological process, cellular component, name, biological source, strand, and sequence for the probe or translational product of the mRNA target of the probe; and (4) a pointer to a next instance of the class “arrayElement,” used for constructing a virtual microarray, as described below. The class “arrayElement” additionally includes two private function members, declared above on lines 16-17: (1) “getNext,” which returns a pointer to a next instance of the class “arrayElement”; (2) “setNext,” which sets the private data member “next” to a supplied value. The class “arrayElement” includes 14 public function members, declared above on lines 20-33, which allow the field values to be retrieved and to be set, as well as a constructor, declared above on line 34.

Next, a declaration for the class “catalogArray” is provided:

```
  1 class catalogArray
  2 {
  3  private:
  4  int rows;
  5  int columns;
  6  arrayHeader header;
  7
  8  stringValCategory molecularFunction;
  9  stringValCategory bioProcess;
10  stringValCategory cellularComp;
11  stringValCategory source;
12  stringValCategory names;
13  stringValCategory sequences;
14
15  arrayElement elements[MAX_ROWS][MAX_COLUMNS];
16
17  bool virtualArray;
18  int vRows;
19  int vCols;
20
21  arrayElement* elDex[MAX_ROWS];
22
23  public:
24  void setRows(int r);
25  int getRows( );
26  void setColumns(int c);
27  int getColumns( );
28
29  void setHeader(arrayHeader h);
30  arrayHeader getHeader( );
31
32  arrayElement* getElement(int row, int column);
33  stringValCategory* getMolecularFunctions( );
34  stringValCategory* getBioProcesses( );
35  stringValCategory* getCellularComps( );
36  stringValCategory* getSources( );
37  stringValCategory* getNames( );
38  stringValCategory* getSequences( );
39
40  void setVirtualArrayMasks(const char* mol, const char* bio,
41  const char* cell, const char* src,
42  const char* name, const char*
                                  seq);
43  void clearVirtualArrayMasks( );
44  void setVirtual( );
45  int getVRows( );
46  int getVCols( );
47  void setCatalog( );
48  bool output(char* file);
49  boot input(char* file);
50  catalogArray( );
51 };
```

The class “catalogArray” includes the following private data members, declared above on lines 4-21: (1) “rows” and “columns,” integers representing the number of rows and columns in the corresponding catalog array; (2) “header,” an instance of the class “arrayHeader” that includes header information for the corresponding catalog array; (3) six instances of the class “stringValCategory,” declared above on lines 8-13, each representing the possible values for, and Boolean mask flags associated with, six different fields in records describing features within the catalog array; (4) “elements,” a two-dimensional array of records representing features in the catalog array; (5) “virtualArray,” a Boolean flag indicating whether or not filters and masking should be applied to produce a virtual array from the catalog array; (6) “vRows” and “vCols,” indications of the number of rows and the number of columns in the currently specified virtual array based on the catalog array; and (7) “elDex,” a one-dimensional array containing pointers to instances of the class “arrayElement,” used to index rows of the currently specified virtual array. The class “catalogArray” includes the following public function members, declared above on lines 24-50: (1) “set” and “get” function members for setting and getting the values of the private data members “rows,” “columns,” and “header,” declared above on lines 24-30; (2) “get” function members that return pointers to array elements and instances of the class “stringValCategory” that describe field values, declared above on lines 32-38; (3) “setVirtualArrayMasks,” a function member that may be repeatedly called to set, as positive masks, various values of the various fields used within records describing features of the catalog array; (4) “clearVirtualArrayMasks,” a function member that clears all masks set by the previously described function; (5) “setVirtual,” a function member that initiates transformation of a catalog array described by an instance of the class “arrraycatalog” into a virtual array, as specified by one or more calls to the above-described function member “setVirtualArrayMasks;” (6) “getVRows” and “getVCols,” function members that return indications of the number of rows and the number of columns in the last row of the currently specified virtual microarray; (7) “setCatalog,” a function member that transforms the currently specified virtual microarray back to the underlying catalog microarray; (8) “output” and “input,” two function members, implementations for which are not provided in the interest of brevity and clarity, that output the contents of an instance of the class “catalogArray” to and from a computer-readable file, object, or other computer-readable data storage entity; and (9) a constructor for the class “catalogArray.”

Next, implementations for various function members of the class “stringValCategory” are provided:

```
  1 int stringValCategory::addVal(const char* val)
  2 {
  3  strcpy (vals[num++], val);
  4  return num;
  5 }
  1 int stringValCategory::getIndex(const char* val)
  2 {
  3  int i;
  4
  5  for (i = 0; i < num; i++)
  6  {
  7  if (!strcmp(vals[i], val)) return i;
  8  }
  9  return −1;
10 }
  1 char* string ValCategory::getString(int index)
  2 {
  3  if (index >= 0 && index < num)
  4  {
  5  return (vals[index]);
  6 }
  7  else return NULL;
  8 }
  1 int stringValCategory::setMaskOn(const char* val)
  2 {
  3  int i;
  4
  5  i = getIndex(val);
  6  if (i >= 0)
  7  {
  8  masks[i] = true;
  9  anyMask = true;
10  }
11  return i;
12 }
  1 int stringValCategory::setMaskOff(const char* val)
  2 {
  3  int i, j;
  4
  5  i = getIndex(val);
  6  if (i >= 0)
  7  {
  8  masks[i] = false;
  9  }
10  anyMask = false;
11  for (j = 0; j < MAX_VALS; j++)
12  if (masks[i] == true) anyMask = true;;
13
14  return i;
15 }
  1 bool stringValCategory::mask(int dex)
  2 {
  3  if (dex >= 0 && dex < num)
  4  {
  5  if (!anyMask) return true;
  6  return masks[dex];
  7  }
  8  else return true;
  9 }
  1 void stringValCategory::clearMasks( )
  2 {
  3  int i;
  4
  5  for (i = 0; i < MAX_VALS; i++) masks[i] = false;
  6  anyMask = false;
  7 }
  1 stringValCategory::stringValCategory( )
  2 {
  3  num = 0;
  4 }
```

The above-provided implementations are, for the most part, straightforward, and do not require detailed commentary. The function member “addVal” simply copies a newly provided text-string value into a list of values. The function member “getIndex” searches the current list of values for a text-string value supplied as argument “val,” and, if such a value is found, returns the index of that text-string value. If no such value is found, a value “−1” is returned. The function member “getString” returns a pointer to a text string corresponding to a supplied index, or the value NULL, if no such text string is currently included in the instances of the class “stringValCategory.” The function member “setMaskOn” sets the Boolean mask value associated with a supplied text-string value so that the text-string value becomes a positive mask for generating virtual microarrays. Note that the data member “anyMask” is set to the value TRUE whenever a Boolean mask flag is set to the value TRUE. The function member “setMaskOff” disables a Boolean mask flag corresponding to a supplied text-string value, and if no other Boolean mask flags are set to TRUE, sets the data member “anyMask” to FALSE, to indicate that the field represented by the instance of the class “stringValCategory” is not involved in the masking and filtering operation used to produce a virtual microarray. The function member “mask” returns the Boolean value of the Boolean mask flag associated with the text-string-value index supplied as argument “dex.” Note that if the index is not valid, a value TRUE is returned. The value TRUE is also returned if the field is not participating within the filtering and masking operations. The final two function members simply clear the Boolean mask flag and initialize an instance of the class “stringValCategory.”

Next, several straightforward representative implementations of member functions of the class “arrayElement” are provided, without further description:

```
  1 int arrayElement::getMolecularFunction( )
  2 {
  3  return molecularFunction;
  4 }
  1 void arrayElement::setMolecularFunction(int dex)
  2 {
  3  molecularFunction = dex;
  4 }
  1 arrayElement::arrayElement( )
  2 {
  3  molecularFunction = −1;
  4  bioProcess = −1;
  5  cellularComp = −1;
  6  name = −1;
  7  source = −1;
  8  strand = −1
  9  sequence = −1;
10  next = NULL;
11 }
```

Next, implementations for various function members of the class “catalogArray” are provided:

```
  1 arrayElement* catalogArray::getElement(int row, int column)
  2 {
  3  arrayElement* nxt;
  4
  5  if (virtualArray)
  6  {
  7  nxt = elDex[row];
  8  while (nxt != NULL && column-- != 0) nxt =
            nxt->getNext( );
  9  return nxt;
10  }
11  else return &(elements[row][column]);
12 }
  1 void catalogArray::setVirtualArrayMasks(const char* mol, const
    char* bio,
  2  const char* cell, const char* src,
  3  const char* name, const char* seq)
  4 {
  5  if (mol != NULL)
  6  {
  7  molecularFunction.setMaskOn(mol);
  8  }
  9  if (bio != NULL)
10  {
11  bioProcess.setMaskOn(bio);
12  }
13  if (cell != NULL)
14  {
15  cellularComp.setMaskOn(cell);
16  }
17  if (src != NULL)
18  {
19  source.setMaskOn(src);
20  }
21  if (name != NULL)
22  {
23  names.setMaskOn(name);
24  }
25  if (seq != NULL)
26  {
27  sequences.setMaskOn(seq);
28  }
29 }
  1 void catalogArray::clearVirtualArrayMasks( )
  2 {
  3  molecularFunction.clearMasks( );
  4  bioProcess.clearMasks( );
  5  cellularComp.clearMasks( );
  6  source.clearMasks( );
  7  names.clearMasks( );
  8  sequences.clearMasks( );
  9 }
  1 void catalogArray::setVirtual( )
  2 {
  3  int rw, cl;
  4  arrayElement* nxt;
  5  arrayElement* prev;
  6  int vRow = 0, vCol = 0;
  7
  8  vRows = 0;
  9  vCols = 0;
10  for (rw = 0; rw < rows; rw++)
11  {
12  for (cl = 0; cl < columns; cl++)
13  {
14  nxt = getElement(rw, cl);
15  if (
16  molecularFunction.mask(nxt->
                    getMolecularFunction( ))
&&
17  bioProcess.mask(nxt->getBioProcess( )) &&
18  cellularComp.mask(nxt->getCellularComp( )) &&
19  source.mask(nxt->getSource( )) &&
20  names.mask(nxt->getName( )) &&
21  sequences.mask(nxt->getSequence( ))
22  )
23  {
24  if (vCol++ == 0)
25  {
26  elDex[vRow] = nxt;
27  nxt->setNext(NULL);
28  }
29  else
30  {
31  prev->setNext(nxt);
32  nxt->setNext(NULL);
33  }
34  prev = nxt;
35  if (vCol == columns)
36  {
37  vCol = 0;
38  vRow++;
39  }
40  }
41  }
42  }
43  if (vCol == 0)
44  {
45  vRows = vRow;
46  vCols = columns;
47  }
48  else
49  {
50  vRows = vRow + 1;
51  vCols = vCol;
52  }
53  virtualArray = true;
54 }
  1 void catalogArray::setCatalog( )
  2 {
  3  clearVirtualArrayMasks( );
  4  virtualArray = false;
  5 }
```

The function member “getElement” returns a pointer to an instance of the class “arrayElement” specified by the supplied row and column. Note that this function member tests the data member “virtualArray,” on line 5, to determine whether or not the instance of the class “catalogArray” is currently being treated as a virtual microarray or as the catalog array. In the former case, the virtual-microarray index “elDex” is used to obtain a pointer to the appropriate virtual-microarray row, and that row is traversed, on line 8, to find the instance of the class “arrayElement” corresponding to the supplied column. If the instance of the class “catalogArray” is being treated as a catalog array, then a pointer to the appropriate element of the catalog array is returned on line 11.

The function member “setVirtualArrayMasks” simply calls corresponding “setMaskOn” function members for the various instances of the class “stringValCategory” that represent various fields within a record that describes a feature within the catalog array. Note that this function can be repeatedly called to specify numerous text-string values for each field to be used as positive masks. The function member “clearVirtualArrayMasks” is straightforward.

The function member “setVirtual” transforms an instance of the class “catalogArray” from a catalog array to a virtual array. This is accomplished by accessing each element of the catalog array, in the nested for-loops of lines 10-42, testing each element to see if it is positively masked, in the if statement of lines 15-22, and if the element is positively masked, including the element into row lists that describe the virtual microarray, in the code of lines 24-40. Finally on lines 43-52, the private data members “vRows” and “vCols” are set according to the number of elements included into the virtual microarray.

Although the present invention has been described in terms of a particular embodiment, it is not intended that the invention be limited to this embodiment. Modifications within the spirit of the invention will be apparent to those skilled in the art. For example, as described above, an almost limitless number of different implementations of a virtualizing microarray may be obtained by varying the programming language, data structures, control structures, modular organizations, and other features and characteristics of the implementation. Moreover, the logic component of a virtualizing microarray may be implemented in hardware, firmware, software, or a combination of two or more of these implementation types. As discussed above, virtualizing microarrays may be employed at any of many different stages and microarray scanning, data processing, and data visualization. Additional functionality may be included to provide more flexible and capable objects and routines. As one example, methods for removing values from instances of the class “stringValCategory” may be included, to allow for editing of field values. As another example, a more flexible and powerful description for category-array features may be developed for describing non-oligonucleotide probes or oligonucleotide probes directed to non-protein-encoding RNAs. XML or other languages may be used to conveniently provide for self-describing catalog microarrays.

Other methods of handling array data can be used as described in U.S. patent application titled “Masking Chemical Arrays”, filed by Peter Webb, Laurakay Bruhn, et al. on the same day as the present application and assigned to Agilent Technologies, Inc. (attorney docket number 10021296-1). The foregoing application is incorporated by reference into the present application.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A virtualizing microarray comprising:
a catalog microarray containing a number of features, each feature containing a type of probe molecule designed to bind a target molecule;
data describing each feature of the catalog microarray; and
logic that generates a virtual microarray from the virtualizing microarray, the virtual microarray comprising data describing a subset of the features of the catalog microarray.

2. The virtualizing microarray of claim 1 wherein the data describing a feature of the catalog microarray includes:
data that identifies a position of the feature within the catalog microarray;
data that identifies the type of probe molecules contained in the feature; and
data that describes the target molecule of the probe molecules contained in the feature.

3. The virtualizing microarray of claim 2 wherein the data that describes the target molecule of the probe molecules contained in the feature includes:
data that describes an immediate target molecule to which the probe molecules bind.

4. The virtualizing microarray of claim 2 wherein the data that describes the target molecule of the probe molecules contained in the feature includes:
data that describes a biological molecule produced by synthesis directed by an immediate target molecule to which the probe molecules bind.

5. The virtualizing microarray of claim 2 wherein the data that describes the target molecule of the probe molecules contained in the feature further includes:
data describing molecular function;
data describing a biological process; and
data describing a cellular component.

6. The virtualizing microarray of claim 1 wherein the logic that generates a virtual microarray from the virtualizing microarray includes:
logic that allows specification of masks related to the data describing each feature of the catalog microarray; and
logic that uses the specified masks to filter the features of the catalog microarray to produce the data describing a subset of the features of the catalog microarray.

7. The virtualizing microarray of claim 1 included in one of:
a microarray scanner;
a microarray-data processing system;
a microarray-data visualization system.

8. The virtualizing microarray of claim 1 further including physically associating the data describing each feature of the catalog microarray with the catalog microarray.

9. The virtualizing microarray of claim 1 wherein the catalog microarray further includes a header that allows the logic to associate data describing each feature of the catalog microarray with a catalog microarray identified by information contained in the header.
